(19) United States
(12) Patent Application Publication
(10) Pub. No.: US 2026/0083590 A1
(43) Pub. Date: Mar. 26, 2026

(54) OPHTHALMIC SURGICAL INSTRUMENTS

(71) Applicant: RAICO INTERNATIONAL, LLC, Westmont, IL (US)

(72) Inventor: Ravi Nallakrishnan, Willowbrook, IL (US)

(21) Appl. No.: 19/109,905

(22) PCT Filed: Sep. 22, 2023
(86) PCT No.: PCT/US2023/074820

§ 371 Date: Mar. 7, 2025

Related U.S. Application Data
(60) Provisional application No. 63/409,020, filed on Sep. 22, 2022.

(51) Int. Cl. A61F 9/007

(52) U.S. Cl. CPC A61F 9/00745; A61B 2217/005

(57) ABSTRACT

An ophthalmic surgical instrument (10, 10A, 10B, 10C, 10D) for use in a phacoemulsification and/or an irrigation and aspiration procedure includes an enlarged emulsification tip (20, 20A, 20B, 200, 20D) for improved capsular bag safety and enhanced efficiency. In one form, the enlarged tip (20, 200) has a pair of opposing convexly curved side walls (22, 220, 26, 260) and a flat bottom wall (28, 280) extending between the convexly curved side walls (22, 220, 26, 260). In another form, the tip (20A) has a convexly 2024/064849 curved top wall (24A) defining a first radius of curvature (r1) , an opposing flat bottom wall (28A) located between two convexly curved side walls (22A, 26A) defining a second radius of curvature (r2). In another form, the tip (20B, 20D) has a convexly curved top surface (40B, 40D) connecting to a needle shaft portion (14B, 14D) and an annular flat end face (SOD).

### PRIORITY

This application claims priority to U.S. provisional application Ser. No. 63/409,020, filed Sep. 22, 2022, the entire contents of which is incorporated herein by reference.

### FIELD OF THE INVENTION

The present invention relates generally to surgical instruments used in ophthalmological surgery and, more particularly, to improved instruments designed for multiple procedures, such as phacoemulsification and irrigation/aspiration surgical procedures.

### BACKGROUND OF THE INVENTION

Phacoemulsification has come to be a technique of choice for the removal of damaged or diseased lenses from the eye. Commonly, such surgery is called for when a patient develops cataracts, a condition in which a portion of the eye lens becomes hard and opaque. Unless the damaged lens is removed and replaced with a properly selected artificial lens, blindness or severely impaired vision will result.

Phacoemulsification is the use of ultrasonic energy to emulsify the damaged lens and aspirate the resulting lens particles from the eye. One of the most significant advantages of the use of phacoemulsification is that the instrument itself is small and can fit through a relatively small incision, resulting in less fluid leakage from the lens capsule of the eye and shorter patient recovery times. It is desirable to limit the amount of ultrasonic energy used as much as possible in order to minimize the risk of damage to eye tissue. Often, the lens nucleus (the hardest portion of the lens) is chopped or split into smaller pieces prior to or during phacoemulsification. Smaller pieces require less energy to emulsify, and this shortens the time during which ultrasonic energy is actually being created by the phacoemulsification apparatus.

Commonly, an infusion sleeve is mounted around the needle to supply irrigating liquids to the eye in order to maintain positive pressure in the eye as the emulsified lens nucleus and fluids are aspirated through the hollow needle.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage is permanent because these cells do not regenerate. One of the benefits of using as small an incision as possible during such surgery is the minimization of leakage of liquid during and after surgery to help prevent tissue collapse, faster healing time, and decreased post-operative astigmatism.

U.S. Pat. No. 7,601,136, hereby incorporated by reference, discloses a phacoemulsification needle and sleeve assembly.

Many phacoemulsification needles and tips are designed for use with handpieces that vibrate the needle longitudinally at relatively low frequencies. In addition to longitudinal vibration, certain handpieces impart a torsional motion to the needle at an oscillation frequency of about one hundred cycles per second. There are also handpieces that provide torsional oscillation of the phacoemulsification tip at frequencies of about 32,000 cycles per second.

Use of the torsional-type handpiece has called for phacoemulsification needle tip designs differing from those used with the longitudinal-type handpiece. For example, needles have been designed with tips that are shaped, swaged and angled to take advantage of the needle motion created by the handpiece.

Many surgeons favor phacoemulsification needles having the straight tip design commonly used with longitudinal handpieces. The great majority of surgeons use longitudinal handpieces rather than the torsional handpieces, often because torsional phacoemulsification equipment is more expensive than longitudinal equipment, and thus these surgeons find themselves unable to take advantage of the enhanced phacoemulsification results claimed by the torsional phacoemulsification systems.

U.S. Pat. No. 10,952,895, hereby incorporated by reference in its entirety, discloses a needle tip in an off-axis position relative to the axis of the aspiration passage extending through the needle body or shaft portion causes eccentric motion or “wobble” during torsional phacoemulsification and improves the efficiency of phacoemulsification while retaining the straight-tip configuration. It has also been found that forming the tip in such an off-axis position also increases the efficiency of phacoemulsification when using a longitudinal handpiece.

Use of an off-axis tip with a longitudinal hand piece appears to desirably create a hybrid type of phacoemulsification motion without using the more complex and expensive torsional phacoemulsification apparatus. The eccentric or wobble type of motion can be imparted to a phacoemulsification needle with no flare at the tip by forming the central aspiration passage within the needle body in an off-axis position. Similar results can be obtained using a straight phacoemulsification needle having an aspiration passage that is formed with a cross-sectional configuration different than the cross-sectional configuration of the needle body itself. These results will be further amplified if the passage is also placed off-axis.

The inventor has found, however, that it may be particularly difficult to cost effectively manufacture aforementioned off-axis type needles. There remains a need for phacoemulsification needles exhibiting improved performance and efficiency that can be easily manufactured in large quantities.

In addition, there remains a need for low cost, multi-purpose surgical instruments that may be safely used for both phacoemulsification and the removal of tissue in the capsular bag in an irrigation and aspiration surgical procedure as set forth in U.S. Pat. No. 7,204,820, which is incorporated herein in its entirety.

The present invention is directed to an improved ophthalmic surgical instrument, having an improved enlarged tip configuration to promote improved emulsification efficiency, improved aspiration, enhanced safety to the posterior lens capsule, the minimization of the transmission of thermal energy to the site during a procedure, and/or lowered cost and improved manufacturability of the needle.

### SUMMARY OF THE INVENTION

The present invention is directed to a multipurpose ophthalmic surgical instrument that may function in both a phacoemulsification procedure and an irrigation and aspiration procedure. In the phacoemulsification procedure, the instrument is particularly suited for use with an associated vibratory surgical handpiece, wherein the handpiece may be configured for torsional (i.e., rotational) ultrasonic movement, as well as linear or longitudinal movement, elliptical, or blended movement, etc.

In accordance with one broad form of the present invention, the instrument includes a hollow needle shaft portion defining (i.e., having) a longitudinal shaft axis extending between proximal and distal ends and having an internal aspiration passage. The instrument includes an enlarged tip joined to (i.e., extending from) the operative, distal end of the needle shaft portion. The enlarged tip defines an open mouth communicating with the aspiration passage. The tip further defines a pair of opposing convexly curved side walls, and a flat bottom wall extending between the convexly curved side walls (as viewed from the exterior of the needle).

In one preferred form of the present invention, the flat bottom wall extends a distance greater than half of the diameter defined by the pair of opposing convexly curved side walls, in a direction perpendicular to a vertical plane containing the longitudinal shaft axis.

In another preferred form of the present invention, the flat bottom wall defines a parabolic surface.

In still another preferred form of the present invention, the enlarged tip further defines a convexly curved top wall opposing said flat bottom wall.

In yet another preferred form of the present invention, the tip further includes a flat top wall opposing the flat bottom wall. Preferably, the flat top wall extends a first distance in a direction perpendicular to a vertical plane containing the longitudinal shaft axis. The flat bottom wall extends a second distance in a direction perpendicular to the aforementioned vertical plane. The first distance is greater than the second distance. Preferably, the first distance is at least 50% greater than the second distance.

In one preferred form of the present invention, the open mouth of the enlarged tip is centered on the longitudinal shaft axis of the needle shaft portion.

According to yet another form of the present invention, the enlarged tip has symmetry about a vertical plane containing the longitudinal shaft axis of the needle shaft portion.

In another preferred form of the present invention, the flat bottom wall extends distally relative to the flat top wall, taken along the longitudinal axis, such that the open mouth is disposed at an acute angle relative to a plane extending normally through the longitudinal axis.

In yet another preferred form of the present invention, the flat top wall extends a greater distance along the longitudinal axis than the flat bottom wall.

According to one preferred form of the present invention, a top frustoconical surface connects between the needle shaft portion and the flat top wall, and a bottom frustoconical surface connects between the needle shaft portion and the flat bottom wall.

According to still another preferred form of the present invention, the flat top wall defines a first thickness, in the vertical direction normal to the longitudinal axis, and the flat bottom wall defines a second thickness (in the same direction) that is greater than the first thickness.

In one form of the present invention, the enlarged tip further defines a convexly curved top wall opposing the flat bottom wall, and the convexly curved top wall defines a first thickness, and the flat bottom wall defines a second thickness that is less than the first thickness.

In accordance with another broad form of the present invention, the instrument includes a hollow needle shaft portion defining a longitudinal shaft axis extending between proximal and distal ends and having an internal aspiration passage. The instrument includes an enlarged tip joined to (i.e., extending from) the operative, distal end of the needle shaft portion. The enlarged tip defines a convexly curved top wall having a first radius of curvature and an opposing flat bottom wall located between two convexly curved side walls defining a second radius of curvature. Preferably, the first radius of curvature is greater than the second radius of curvature.

According to one form of the present invention, the open mouth is centered on the longitudinal shaft axis. Preferably, the enlarged tip has symmetry about a vertical plane containing the longitudinal shaft axis.

In another preferred form of the present invention, the flat bottom wall extends distally relative to the convexly curved top wall, along the longitudinal axis, such that the open mouth is disposed at an acute angle relative to a plane extending normally through the longitudinal axis. Preferably, the convexly curved top wall extends a greater distance along the longitudinal axis than the flat bottom wall.

According to still another form of the present invention, a top frustoconical surface connects between the needle shaft portion and the convexly curved top wall, and a bottom frustoconical surface connects between the needle shaft portion and the flat bottom wall.

According to another form of the present invention, the convexly curved top wall defines a first thickness (in the vertical direction perpendicular to the shaft axis), and the flat bottom wall defines a second thickness in the same direction that is greater than the first thickness.

In accordance with still another broad form of the present invention, the instrument includes a hollow needle shaft portion defining a longitudinal shaft axis extending between proximal and distal ends and having an internal aspiration passage. The instrument includes an enlarged tip joined to the operative, distal end of the needle shaft portion. The enlarged tip defines an enlarged tip defining a convexly curved top surface connecting to the needle shaft portion, and the enlarged tip defines an annular flat end face disposed at an acute angle relative to a plane extending normally through the longitudinal shaft axis. Preferably, the acute angle is about 5 degrees (+/−3 degrees).

In one preferred form of the present invention, the open mouth is centered on a tip axis that is coincident with the longitudinal shaft axis.

In one form of the present invention, the enlarged tip defines a bottom frustoconical surface connecting to the needle shaft portion.

In still another preferred form of the present invention, the bottom frustoconical surface extends distally relative to the convexly curved top surface, taken along a tip axis, such that the open mouth is disposed at an acute angle relative to a plane extending normally through the tip axis.

According to one preferred form of the present invention, the instrument includes a bend in the needle shaft portion that is located proximate to the enlarged tip.

In another broad form of the present invention, the instrument is provided with a vibratory handpiece removably attached to a proximal end of the needle shaft portion.

### BRIEF DESCRIPTION OF THE DRAWINGS

In the accompanying drawings forming part of the specification, in which like numerals are employed to designate like parts throughout the same,

FIG. 1 is an isometric view, from the front and right side, of a first illustrated embodiment of an ophthalmic surgical instrument of the present invention shown assembled with a vibratory handpiece shown diagrammatically;

FIG. 2 is a front elevational view of the surgical instrument shown in FIG. 1;

FIG. 3 is a greatly enlarged, fragmentary, right-side elevational view of the operative tip portion of the instrument shown in FIG. 1, the left-side being identical thereto;

FIG. 3A is a greatly enlarged, fragmentary, isometric view from the front and right side of the instrument shown in FIG. 1;

FIG. 4 is an isometric view, from the front and right side, of a second illustrated embodiment of an ophthalmic surgical instrument of the present invention;

FIG. 5 is a front elevational view of the surgical instrument shown in FIG. 4;

FIG. 6 is a greatly enlarged, fragmentary, right-side elevational view of the operative tip portion of the instrument shown in FIG. 4, the left-side being identical thereto;

FIG. 7 is a greatly enlarged, fragmentary, cross-sectional view of the operative tip portion of either the instruments of FIG. 1 or 4, taken along a vertical plane extending through a central longitudinal axis of the body and tip of the instrument;

FIG. 7A is a fragmentary, bottom plan view of the operative tip portion of the instrument shown in FIG. 4;

FIG. 8 is a right side elevational view of a third illustrated embodiment of an ophthalmic surgical instrument of the present invention, the left-side being identical thereto;

FIG. 9 is a fragmentary, greatly enlarged isometric view, from the front and right side, of the operative portion of the surgical instrument shown in FIG. 8;

FIG. 10 is a greatly enlarged, right-side elevational view of a fourth illustrated embodiment of an ophthalmic surgical instrument of the present invention, the left-side being identical thereto;

FIG. 11 is a greatly enlarged, front elevation view of the instrument shown in FIG. 10;

FIG. 12 is a greatly enlarged isometric view, from the front and right side, of the surgical instrument shown in FIG. 10;

FIG. 13 is a greatly enlarged isometric view, from the bottom and right side, of the surgical instrument shown in FIG. 10;

FIG. 14 is a greatly enlarged, cross-sectional view of instrument of FIG. 10, taken along a vertical plane extending through a central longitudinal axis of the body and tip of the instrument;

FIG. 15 is a greatly enlarged, right-side elevational view of a fifth illustrated embodiment of an ophthalmic surgical instrument of the present invention;

FIG. 16 is a greatly enlarged, front elevation view of the instrument shown in FIG. 15;

FIG. 17 is a greatly enlarged isometric view, from the front and right side, of the surgical instrument shown in FIG. 15;

FIG. 18 is a greatly enlarged isometric view, from the bottom and right side, of the surgical instrument shown in FIG. 15; and

FIG. 19 is a greatly enlarged, cross-sectional view of instrument of FIG. 15, taken along a vertical plane extending through a central longitudinal axis of the body and tip of the instrument.

### DETAILED DESCRIPTION OF THE PREFERRED EMBODIMENTS

While the present invention is susceptible of embodiment in various forms, there are shown in the drawings and will hereinafter be described the presently preferred embodiments, with the understanding that the present disclosure should be considered as an exemplification of the invention, and is not intended to limit the broadest forms of the invention to only the specific embodiments illustrated.

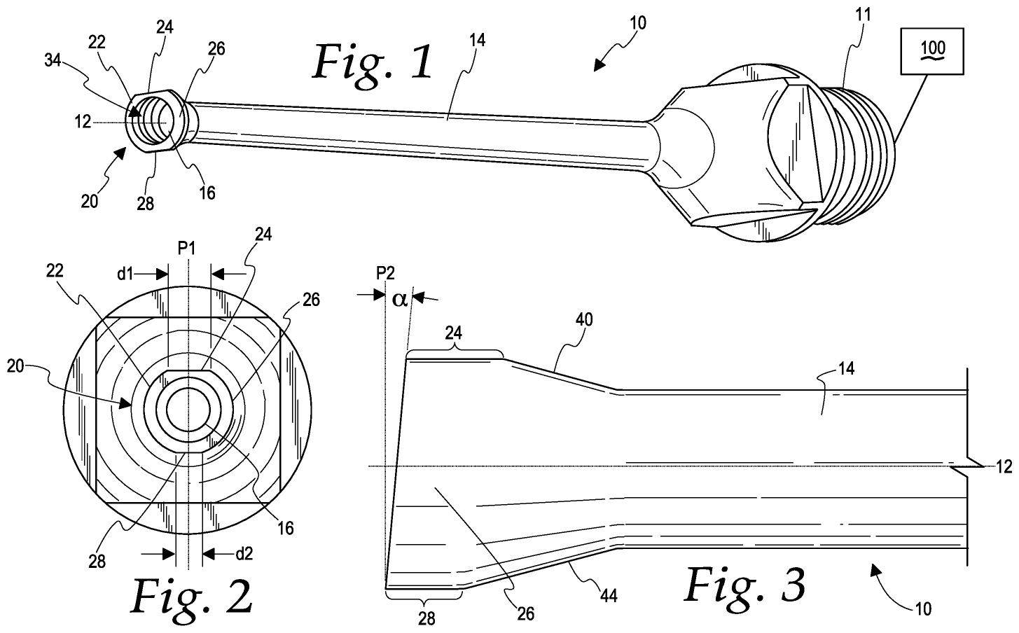

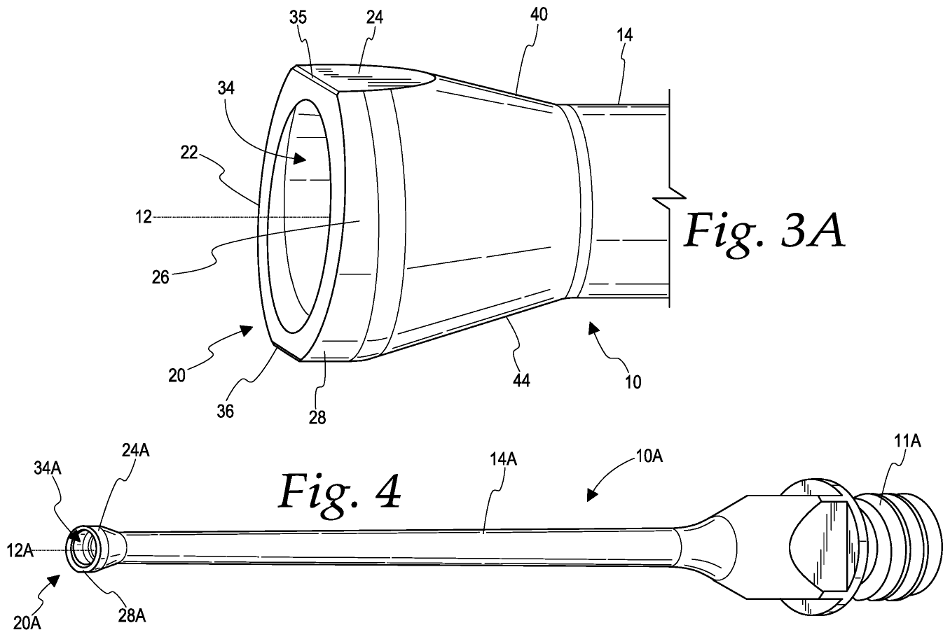

A first illustrated embodiment of an ophthalmic surgical instrument according to the present invention is shown in FIGS. 1-3 and 7, wherein the instrument 10 includes an elongate needle shaft portion 14 having a threaded proximal end 11 (FIG. 1) for being connected to mating threads in an irrigation/aspiration system or handpiece 100 and/or vibratory system or handpiece 100 for performing a phacoemulsification procedure (shown diagrammatically in FIG. 1 only) and defining a central, longitudinal shaft axis 12 (FIGS. 1 and 3). The vibratory handpiece 100 may be any one of a longitudinally-vibrating handpiece, a torsionally-vibrating handpiece, an elliptically-vibrating handpiece, and/or a blend thereof. Other commercially available handpieces are contemplated. The shaft portion 14 defines an internal aspiration passage 16 (FIG. 2), through which aspiration is effected during use of the instrument when the proximal end 11 is subjected to a vacuum source. Only a distal portion of the instrument 10 is illustrated in many of the accompanying figures, and it will be understood that the proximal end 11 of the instrument disclosed herein may have a variety of means or structures suited for being attached to a hand piece, such as by mating screw threads, clamps, locks, friction fitting, etc. Suitable proximal end structures for mating with handpieces are described in U.S. Pat. No. 8,764,782, the entirety of which is incorporated herein by reference.

With reference to FIGS. 1 and 2, the present surgical instrument 10 further comprises an operative, enlarged emulsification tip 20 joined to a distal end of the needle shaft portion 14, opposite of the proximal end 11. Notably, the emulsification tip 20 has a generally, truncated circular cross-sectional configuration, when viewed in a plane extending perpendicular to the longitudinal shaft axis 12 (i.e., as the instrument 10 is viewed in FIG. 2), and includes a pair of opposing convexly curved side walls 22, 26, a flat top wall 24, and an opposing flat bottom wall 28 extending between the convexly curved side walls 22, 26—all together defining a perimeter of an open mouth 34. The open mouth 34 preferably defines an internal surface that is circular in cross-section (as viewed in FIG. 2) for ease of manufacture of the tip 20, and it communicates with, or connects to, the aspiration passage 16 of the needle shaft portion 14 to facilitate aspiration of emulsified tissues through the instrument 10. The mouth 34 is centered on the longitudinal axis 12.

With reference to FIG. 2, it can be seen that the flat top wall 24 extends a first distance d1 in a direction perpendicular to a vertical plane P1 containing the longitudinal shaft axis 12. Furthermore, the flat bottom wall 28 extends a second distance d2 in the same direction perpendicular to the vertical plane P1. The first distance d1 is greater than the second distance d2. In the preferred embodiment, the length of the first distance d1 is at least 50% greater in length than the distance d2. It can further be seen that the tip 20 has symmetry about a vertical plane P1 (extending up and down in FIG. 2) which contains the longitudinal axis 12.

As can be seen in FIG. 7, the flat top wall 24 defines a first thickness T1 in the direction along a vertical plane P2 that is perpendicular to the longitudinal axis 12. The first thickness T1 of the top wall 24 is substantially thinner than a second thickness T2 the flat bottom wall 28 in the same direction. The differentiation of thicknesses of the walls 24 and 28 enhance vibration during a phacoemulsification procedure in any of torsional, longitudinal, elliptical, and/or blended ultrasonic movement of a vibratory handpiece 100 connected to the instrument 10. The wobble effect preferably occurs in all three orthogonal planes (X-Y-Z) during operation of the instrument 10 in longitudinal and/or torsional vibratory modes. Preferably, the top wall 24 has a nominal thickness T1 at its geometric center of between about 0.01 mm and about 0.6 mm, and more preferably about 0.10 mm. Preferably, the flat bottom wall 28 has a nominal thickness T2 at its geometric center of between about 0.01 mm and about 0.6 mm, and more preferably about 0.16 mm. In the illustrated embodiment of the instrument 10 of FIG. 7, the second thickness T2 is at least 50% greater than the first thickness T1 to impart the desired wobble effect during operation of the instrument 10. Preferably, the diameter of the mouth 34 of the enlarged tip 20 is between about 0.5 mm and about 2.5 mm.

In the illustrated first embodiment of the instrument 10, the convexly-curved side or lateral walls 22, 26 provide enhanced safety for the posterior capsule of the eye while the flat top wall 24 and flat bottom wall 28 are dimensioned to provide the advantageous wobble effect without requiring the offset boring of axes of the tip 20 and the needle shaft portion 14, as required by instruments of the prior art.

With reference to FIG. 3, the top flat wall 24 is connected to the needle shaft portion 14 by a top frustoconical surface 40. The flat bottom wall 28 is connected to the needle shaft portion 14 by a bottom frustoconical surface 44. It can be seen that the flat top wall 24 extends a length or distance along the longitudinal shaft axis 12 that is substantially greater than a length or distance of the flat bottom wall 28.

Still referring to FIG. 3, the flat bottom wall 28 extends distally relative to the flat top wall 24, along the longitudinal axis 12, such that the open mouth 34 is disposed at an acute angle a relative to the plane P2 extending normally through the longitudinal axis. The acute angle a may be between about 0 degrees and about 90 degrees, and angle a is more preferably between about 5 degrees and about 15 degrees, relative to the plane P2.

The mouth 34 defines a central, longitudinal mouth axis, which is not numbered because it is substantially coincident with the longitudinal axis 12 of the needle shaft portion 14 to enhance manufacturability of the instrument 10 while simultaneously providing an advantageous emulsifying or chopping action of the walls 24 and 28 (which are varied in two orthogonal dimensions) during vibratory operation of the instrument 10. The coaxial alignment of the tip and body axes permit a single milling, drilling, or other material removal process to be used to significantly reduce manufacturing complexity and costs compared to prior art instrument configurations.

Referring again to FIG. 7, the interior of the instrument 10 includes smooth, opposing transition points 46, 48, between the mouth 34 of the enlarged tip 20 and the needle shaft portion 14 to avoid, or at least minimize, repulsion of aspirated material as well as provide a second point of emulsification during operation of the instrument 10. The transition from the enlarged tip 20 to the narrower needle shaft portion 14, preferably from about 0.9 mm internal diameter to about 0.8 mm internal diameter, is believed to enhance aspiration rate and the vacuum developed during operation of the instrument 10 to higher than that of the setting on the associated handpiece controller.

A desirable feature of the present instrument is the absence of sharp edges on the exterior of the instrument 10. The forward, distal edges of the tip 20 are preferably rounded and smooth, without sharp edges. There are preferably no sharp edges on the outer periphery of the tip 20. Referring to FIG. 3A, the mouth 34 to the tip 20 includes rounded forward edges 35 and 36 of the flat top and bottom walls 24 and 28, respectively, to provide improved safety to the posterior capsule during operation of the instrument 10. The geometry of the flat top and bottom walls 24 and 28 allows the surgeon to sculpt, groove, and/or trench the nucleus of the eye with increased safety of the rounded forward edges or bevels 35 and 36.

The specific configuration of the present instrument 10 can be varied depending upon intended use. The needle shaft portion 14 can be straight as in the illustrated first embodiment, or the needle shaft portion 14 may be bent to a Kelman-style configuration for effecting chopping during a surgical procedure. Alternatively, the needle shaft portion 14 can be bent to an Akahoshi-style (Reverse Kelman Bend) for pre-chopping during a procedure. Other bent configurations are contemplated.

The outer surface of the tip 20 may have a sandblasted finish to eliminate or at least reduce the potential for sharp edges for improved safety of the instrument 10 in the eye. Such sandblasting or other conventional or non-conventional finishing methods may be applied to the instrument embodiments discussed below.

A second embodiment of an ophthalmic surgical instrument according to the present invention is shown in FIGS. 4-7, designated by the numeral 10A, and functions similarly to the first illustrated embodiment of the instrument 10 as previously described in FIGS. 1-3. The numbered features of the second embodiment of the instrument 10A illustrated in FIGS. 4-7 are analogous to features of the first embodiment of the instrument 10 that share the same number, but without the suffix “A”. The second embodiment of the surgical instrument 10A includes the same basic structures of a needle shaft portion 14A defining a central, longitudinal shaft axis 12A and an aspiration passage 16A extending through the shaft portion 14A. The instrument 10A includes a proximal end 11A with attachment means, as discussed above, for being removably connected to an irrigation/aspiration and/or vibratory handpiece. An enlarged emulsification tip 20A extends from the distal end of the shaft portion 14A. The tip 20A differs in that it includes a continuously convexly curved top wall 24A defining a first radius of curvature r1, and further includes an opposing flat bottom wall 28A located between two convexly curved side walls 22A, 26A defining a second radius of curvature r2. The walls 22A, 24A, 26A, and 28A collectively define a perimeter of an open mouth 34A, which has a circular interior surface or cross-sectional shape which is centered on the longitudinal shaft axis 12A, and which has symmetry about a vertical plane P1 containing the shaft axis 12A (FIG. 5).

As can be seen in FIG. 5, the first radius of curvature r1 of the curved top wall 24A is greater (i.e., longer) than the radius of curvature r2 of the two convexly curved side walls 22A, 26A defining a second radius of curvature r2. The tip 20A of the instrument 20A significantly differs from the prior embodiment of the instrument 10 in that the enhanced wobble effect during vibratory excitation of the instrument 10A is formed by the continuous thinning of the curved geometry of the top wall 24A.

As can be seen in FIG. 7, the curved top wall 24A defines a first thickness T1 in the direction along a vertical plane P2 that is perpendicular to the longitudinal axis 12A. The first thickness T1 of the top wall 24A is substantially thinner than a second thickness T2 the flat bottom wall 28A in the same direction. The differentiation of thicknesses of the walls 24A and 28A enhance vibration during a phacoemulsification procedure in any of torsional, longitudinal, elliptical, and/or blended ultrasonic movement of a vibratory handpiece connected to the instrument 10A. Preferably, the top wall 24A has a nominal thickness T1 at its geometric center of between about 0.01 mm and about 0.6 mm, and more preferably about 0.10 mm. Preferably, the flat bottom wall 28A has a nominal thickness T2 at its geometric center of between about 0.01 mm and about 0.6 mm, and more preferably about 0.16 mm. In the illustrated embodiment of the instrument 10A of FIG. 7, the second thickness T2 is at least 50% greater than the first thickness T1 to impart the desired wobble effect during operation of the instrument 10A. Preferably, the diameter of the mouth 34A of the enlarged tip 20A is between about 0.5 mm and about 2.5 mm.

In the illustrated second embodiment of the instrument 10A, the convexly-curved top wall 24A and side or lateral walls 22A, 26A provide enhanced safety for the posterior capsule of the eye while still providing the advantageous wobble effect without requiring offset formation of axes of the tip 20A and the needle shaft portion 14A, as required by the prior art.

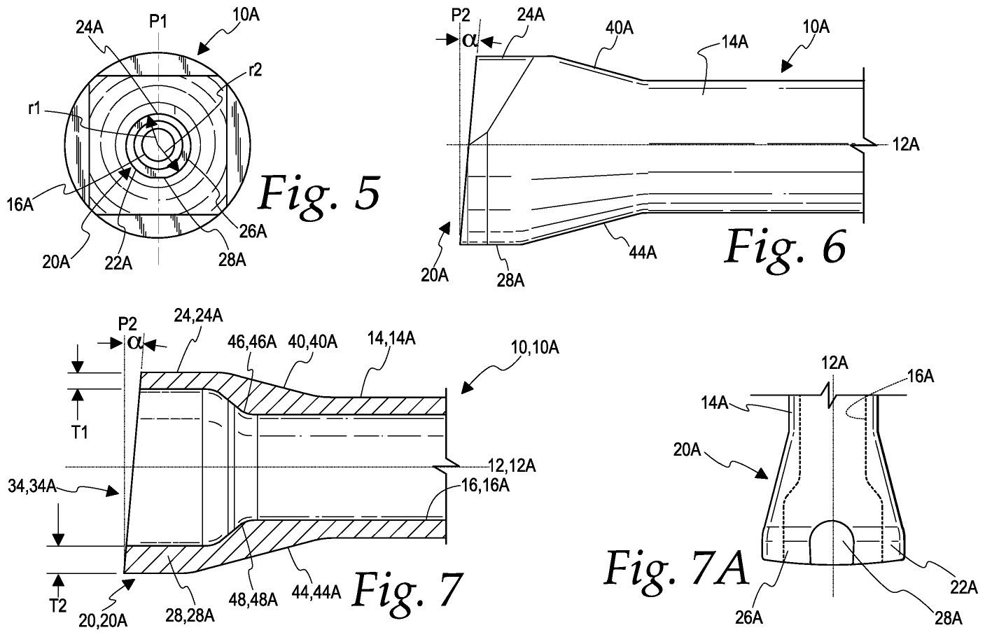

With reference to FIG. 6, the convexly-curved top wall 24A of the enlarged tip 20A is connected to the needle shaft portion 14A by a top frustoconical surface 40A. The flat bottom wall 28A is connected to the needle shaft portion 14A by a bottom frustoconical surface 44A. It can be seen that the top wall 24A extends a length or distance along the longitudinal shaft axis 12A that is substantially greater than a length or distance of the flat bottom wall 28A in the same direction.

Still referring to FIG. 6, the flat bottom wall 28A extends distally (i.e., away from the proximal end 11A of the needle shaft portion 14A) relative to the curved top wall 24A, along the longitudinal axis 12A, such that the open mouth 34A is disposed at an acute angle a relative to the plane P2 extending normally through the longitudinal axis 12A. The acute angle α may be between about 0 degrees and about 90 degrees, and the angle a is more preferably between about 5 degrees and about 15 degrees, relative to the plane P2.

The specific configuration of the instrument 10A can be varied depending upon intended use. The needle shaft portion 14A can be straight as in the illustrated second embodiment of the instrument 10A, or the needle shaft portion 14A may be bent to a Kelman-style configuration for effecting chopping during a surgical procedure. Alternatively, the needle shaft portion 14A can be bent to an Akahoshi-style (Reverse Kelman Bend) for pre-chopping during a procedure. Other bent configurations are contemplated. The inventor has found that the rounded edge or bevel of the flat bottom wall 28A permits the surgeon to sculpt, groove, and/or trench the nucleus of the eye with increased safety. The instrument 10A is drilled a first time to create the aspiration passage 16A and a second time partially to create the larger opening of the mouth 34A for more efficiency and weight imbalance during operation of the instrument 10A.

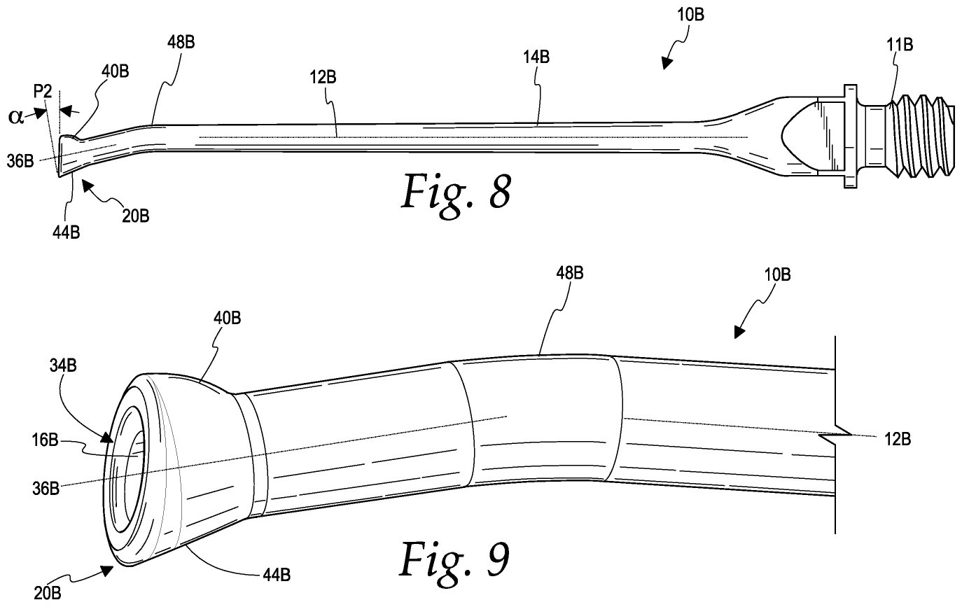

A third embodiment of an ophthalmic surgical instrument according to the present invention is shown in FIGS. 8 and 9 and is designated by the numeral 10B. The instrument 10B functions similarly to the first illustrated embodiment of the instrument 10 as previously described in FIGS. 1-3, and 7. The numbered features of the third embodiment of the instrument 10B illustrated in FIGS. 8 and 9 are analogous to features of the first embodiment of the instrument 10 that share the same number (without the suffix “B”). The third embodiment of the surgical instrument 10B differs from the aforementioned first illustrated embodiment in that the instrument 10B includes an emulsification tip 20B with a partially-spherical, convexly curved top surface 40B connecting to the needle shaft portion 14B and an opposite, bottom frustoconical surface 44B connecting to the needle shaft portion 14B. The hybrid geometry of the enlarged tip 20B having the partially-spherical top surface 40B provides enhanced safety to the lens capsule, while the bottom frustoconical surface 44B provides better overall efficiency when compared to prior art phacoemulsification needles.

With reference to FIG. 8, the needle shaft portion 14B includes a bend 48B located proximate to the enlarged tip 20B. The specific configuration of the present instrument 10B can be varied depending upon intended use. The needle shaft portion 14B can be straight as in the illustrated first and second embodiments of the instrument 10 and 10A, respectively, or the needle shaft portion 14B may be bent to a Kelman-style configuration for effecting chopping during a surgical procedure. Alternatively, the needle shaft portion 14B can be bent to an Akahoshi-style (Reverse Kelman Bend) for pre-chopping during a procedure. The angle of the bend 48B in the shaft portion 14B is preferably between 0 degrees and 60 degrees, relative to the shaft axis 12B. Other bent configurations are contemplated. Importantly, the tip 20B defines a tip axis 36B that is coincident with the longitudinal axis 12B (if the longitudinal axis 12B were to be extended through the bend 48B along the geometric center of the shaft portion 14B) to enhance manufacturability of the instrument 10B while simultaneously providing an advantageous emulsifying or chopping action of the opposite surfaces 40B and 44B during vibratory operation of the instrument 10B. The coaxial alignment of the tip and body axes permit a single milling, drilling, or other material removal process to be used to create the aspiration passage 16B to significantly reduce manufacturing complexity and costs compared to prior art instrument configurations.

Referring to FIG. 8, the bottom surface 44B extends distally relative to the top surface 40B, along the tip axis 36B, such that the open mouth 34B is disposed at an acute angle a relative to the plane P2 extending normally through the tip axis 36B. The acute angle a may be between about 0 and about 90 degrees, and the angle a is more preferably between about 5 degrees and about 15 degrees, relative to the plane P2.

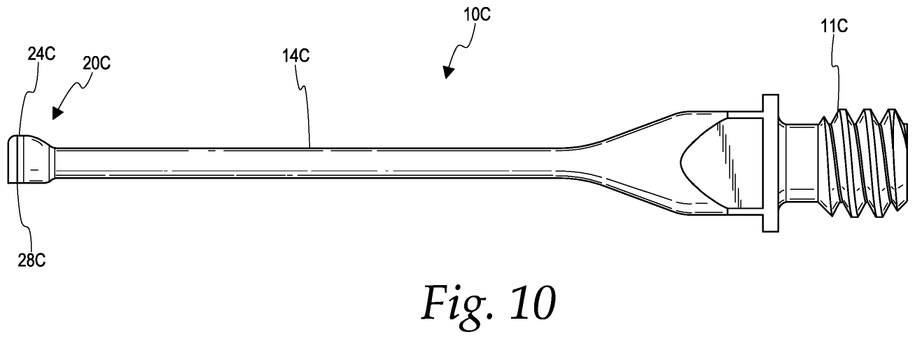

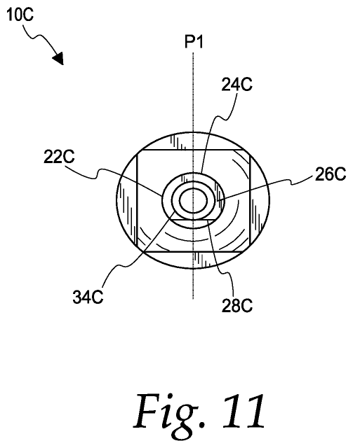

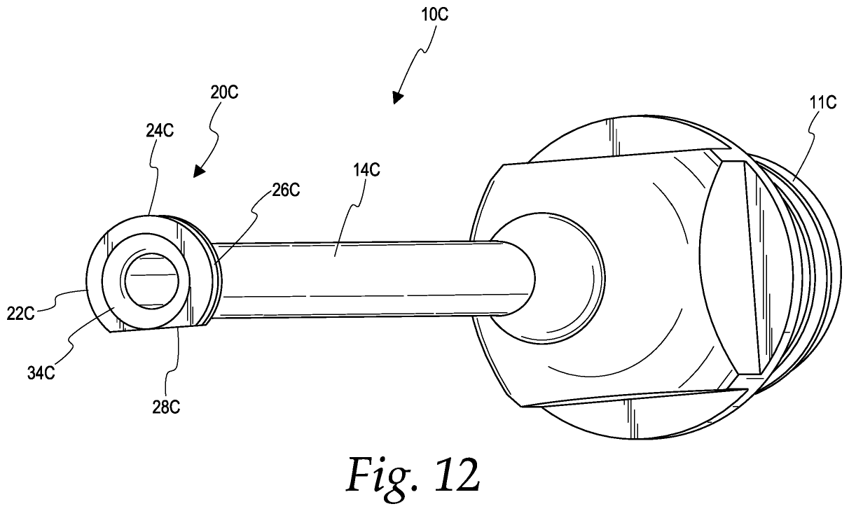

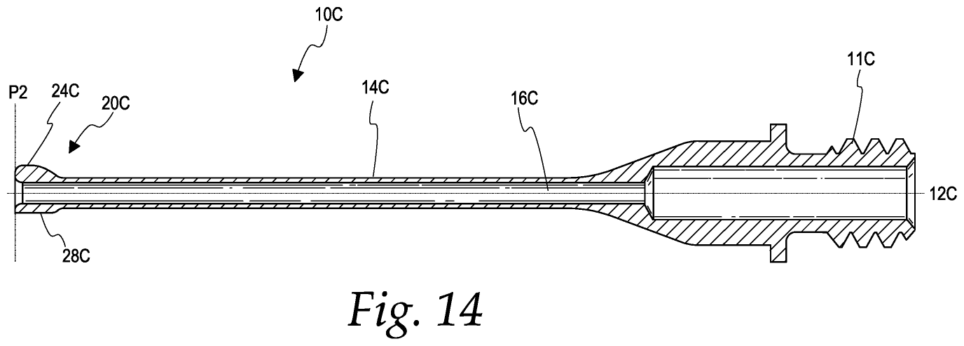

A fourth, presently most preferred, embodiment of an ophthalmic surgical instrument according to the present invention is shown in FIGS. 10-14 and is designated by the numeral 10C. The instrument 10C functions similarly to the first illustrated embodiment of the instrument 10 as previously described in FIGS. 1-3, and 7. The numbered features of the fourth embodiment of the instrument 10C illustrated in FIGS. 10-14 are analogous to features of the first embodiment of the instrument 10 that share the same number (without the suffix “C”). The fourth embodiment of the surgical instrument 10C differs from the aforementioned first illustrated embodiment in that the instrument 10C includes an emulsification tip 20C with a different truncated circular cross-sectional configuration, when viewed in a plane extending perpendicular to the longitudinal shaft axis 12C (i.e., as the instrument 10C is viewed in FIG. 11), and includes a pair of opposing convexly curved side walls 22C, 26C, a convexly curved top wall 24C, and an opposing flat bottom wall 28C extending between the convexly curved side walls 22C, 26C-all together defining a perimeter of an open mouth 34C. The open mouth 34C preferably defines an internal surface that is circular in cross-section (as viewed in FIG. 11) for ease of manufacture of the tip 20C, and it communicates with, or connects to, the aspiration passage 16C of the needle shaft portion 14C to facilitate aspiration of emulsified tissues through the instrument 10C. The mouth 34C is centered on the longitudinal axis 12C. The inventor has found the geometry of the tip 20C having the convexly curved top wall 24C provides enhanced safety to the lens capsule, while the bottom flat wall 28C provides better overall efficiency when compared to prior art phacoemulsification needles which accommodates the user for sculpting and trenching of the natural lens. Finite element analysis of torsional and/or longitudinal vibratory modes at 30 and 40 kHz of the instrument 10C found that the tip 20C configuration exhibited the most efficiency compared to needles of the prior art.

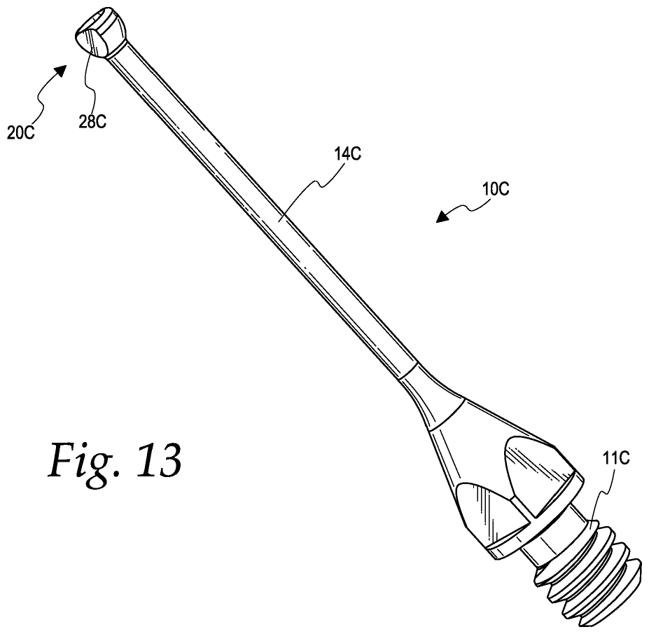

With reference to FIG. 11, it can be seen that the flat bottom wall 28C extends a distance more than half the diameter of the tip 20C in a direction perpendicular to the vertical plane P1. The flat bottom wall 28C defines a defines a parabolic surface, as can be seen in FIG. 13 and has a thickness that is less than half a thickness of the opposing top wall 24C, in the direction along plane P2 that runs perpendicular to the longitudinal axis 12C.

With reference to FIG. 10, the needle shaft portion 14C includes no bend located proximate to the enlarged tip 20C. The specific configuration of the present instrument 10C can be varied depending upon intended use. The needle shaft portion 14C can be straight as illustrated, or the needle shaft portion 14C may be bent to a Kelman-style configuration for effecting chopping during a surgical procedure. Alternatively, the needle shaft portion 14C can be bent to an Akahoshi-style (Reverse Kelman Bend) for pre-chopping during a procedure. Other bent configurations are contemplated.

Referring to FIG. 11, the top wall 24C and the bottom wall 28C terminate at the same location, along the axis 12C. In some embodiments, it may be preferable for the bottom wall 28C to extend beyond the top wall 24C such that the open mouth 34C is disposed at an acute angle a relative to the plane P2 extending normally through the axis 12C (not illustrated). The acute angle a may be between about 0 and about 90 degrees, and the angle a is more preferably between about 5 degrees and about 15 degrees, relative to the vertical plane P2.

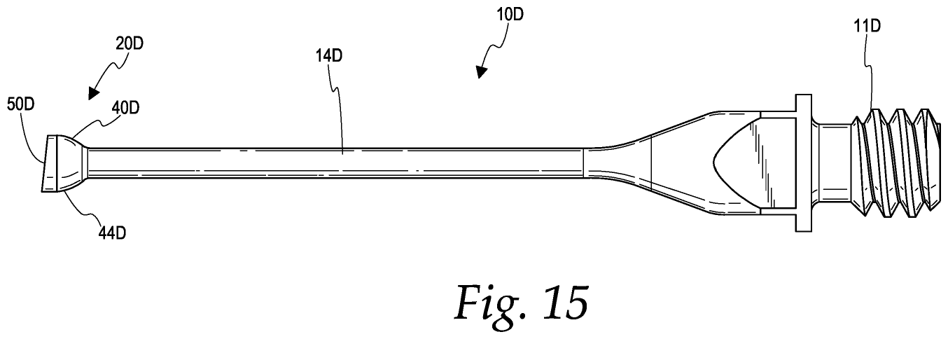

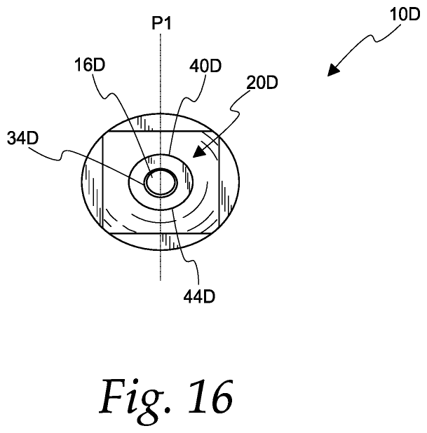

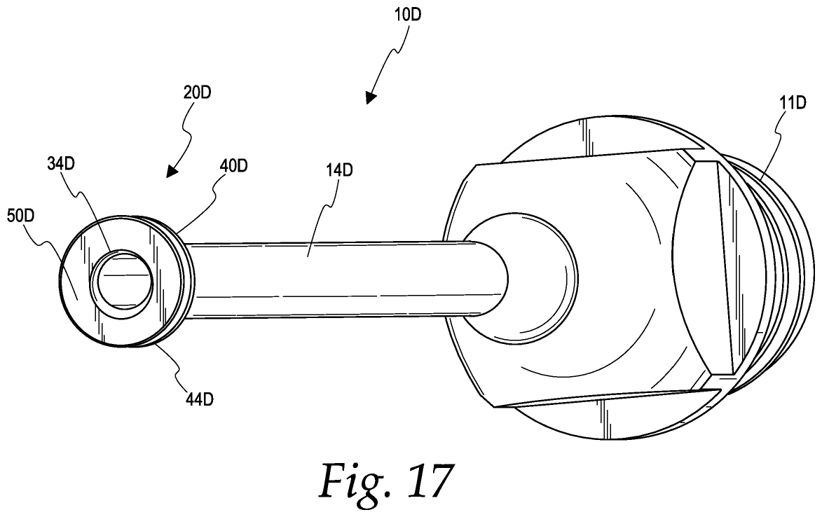

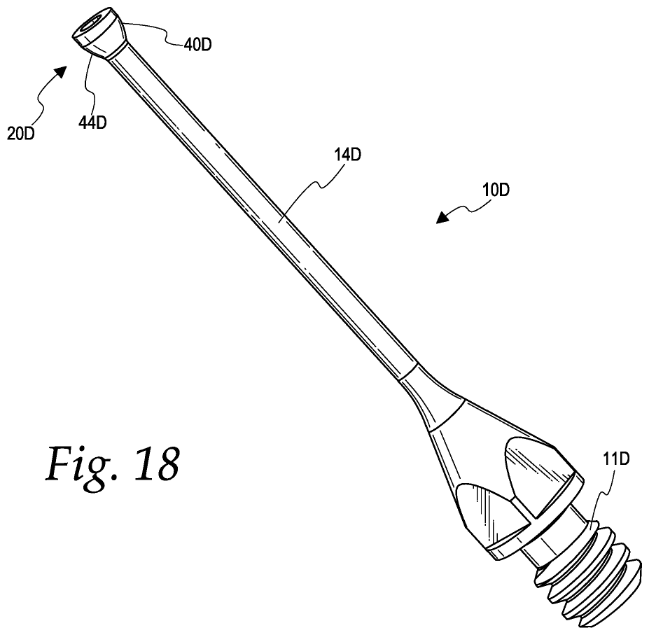

A fifth embodiment of an ophthalmic surgical instrument according to the present invention is shown in FIGS. 15-19 and is designated by the numeral 10D. The instrument 10D functions similarly to the third illustrated embodiment of the instrument 10B as previously described in FIGS. 8 and 9. The numbered features of the fifth embodiment of the instrument 10D illustrated in FIGS. 15-19 are analogous to features of the third embodiment of the instrument 10 that share the same number (without the suffix “D”). The fifth embodiment of the surgical instrument 10D differs from the aforementioned third illustrated embodiment in that the instrument 10D includes an emulsification tip 20D includes an emulsification tip 20D with a partially-spherical, convexly curved top surface 40D connecting to the needle shaft portion 14D and an opposite, bottom partially-spherical, convexly curved bottom surface 44D connecting to the needle shaft portion 14D. The tip 20D terminates in an annular, flat end face 50D. The aspiration passage 16D is preferably formed with a double pass drill.

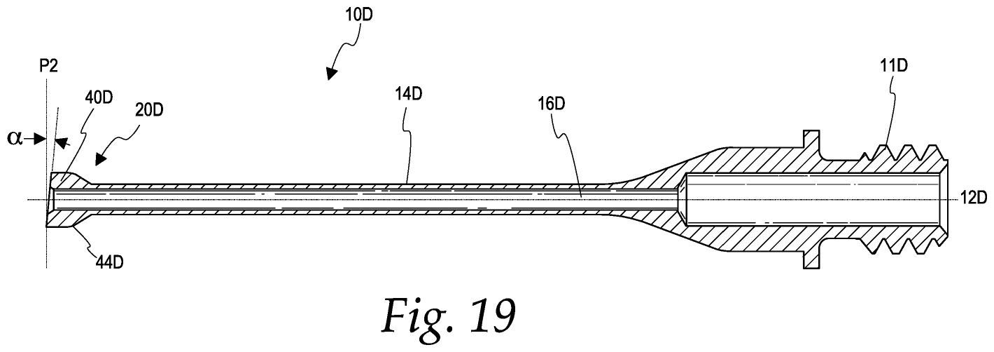

Referring to FIGS. 15 and 19, the bottom surface 44D extends distally relative to the top surface 40D, along the axis 12D, such that the open mouth 34D and the flat, annular end face 50D are each disposed at an acute angle a relative to the plane P2 extending normally through the axis 12. The acute angle a may be between about 0 and about 90 degrees, and the angle a is more preferably about 5 degrees, relative to the plane P2. The beveled geometry of the enlarged tip 20D provides enhanced safety to the lens capsule and better overall efficiency when compared to prior art phacoemulsification needles. The inventor has found the beveled geometry of the tip 20D having the flat end face 50D also provides better overall efficiency when compared to prior art phacoemulsification needles which, unlike prior art needles, accommodates the user for sculpting and trenching of the natural lens. Finite element analysis of torsional and/or longitudinal vibratory modes at 30 and 40 kHz of the instrument 10D found that the tip 20C configuration exhibited the improved efficiency compared to needles of the prior art.

With reference to FIG. 15, the needle shaft portion 14D includes no bend located proximate to the enlarged tip 20D. The specific configuration of the present instrument 10D can be varied depending upon intended use. The needle shaft portion 14D can be straight as illustrated, or the needle shaft portion 14D may be bent to a Kelman-style configuration for effecting chopping during a surgical procedure. Alternatively, the needle shaft portion 14D can be bent to an Akahoshi-style (Reverse Kelman Bend) for pre-chopping during a procedure. Other bent configurations are contemplated.

From the foregoing, it will be observed that numerous modifications and variations can be effected without departing from the true spirit and scope of the novel concept of the present invention. It is to be understood that no limitation of the broadest concepts with respect to the specific embodiments illustrated herein is intended or should be inferred. The disclosure is intended to cover, by the appended claims, all such modifications as fall within the scope of the claims.

1. An ophthalmic surgical instrument, said instrument comprising:
a hollow needle shaft portion defining a longitudinal shaft axis and having an internal aspiration passage; and
an enlarged tip joined to a distal end of said needle shaft portion,
said enlarged tip defining an open mouth communicating with said aspiration passage, said enlarged tip defining a pair of opposing convexly curved side walls, said enlarged tip further defining a flat bottom wall extending between said convexly curved side walls.

2. The ophthalmic surgical instrument in accordance with claim 1, wherein said flat bottom wall extends a distance greater than half of a diameter defined by said pair of opposing convexly curved side walls, in a direction perpendicular to a vertical plane containing said longitudinal shaft axis.

3. The ophthalmic surgical instrument in accordance with claim 1, wherein said flat bottom wall defines a parabolic surface.

4. The ophthalmic surgical instrument in accordance with claim 1, wherein said enlarged tip further defines a convexly curved top wall opposing said flat bottom wall.

5. The ophthalmic surgical instrument in accordance with claim 1, wherein said enlarged tip further defines a flat top wall opposing said flat bottom wall, said flat top wall extending a first distance in a direction perpendicular to a vertical plane containing said longitudinal shaft axis, and said flat bottom wall extending a second distance in a direction perpendicular to said vertical plane, said first distance is greater than said second distance.

6. The ophthalmic surgical instrument in accordance with claim 5, wherein said first distance is at least 50% greater than said second distance.

7. The ophthalmic surgical instrument in accordance with claim 1, wherein said open mouth is centered on said longitudinal shaft axis.

8. The ophthalmic surgical instrument in accordance with claim 1, wherein said enlarged tip has symmetry about a vertical plane containing said longitudinal shaft axis.

9. The ophthalmic surgical instrument in accordance with claim 1, wherein said enlarged tip further defines a flat top wall opposing said flat bottom wall, and wherein said flat bottom wall extends distally relative to said flat top wall, along said longitudinal axis, such that said open mouth is disposed at an acute angle relative to a plane extending normally through said longitudinal axis.

10. The ophthalmic surgical instrument in accordance with claim 1, wherein said enlarged tip further defines a flat top wall opposing said flat bottom wall, and wherein said flat top wall extends a greater distance along said longitudinal axis than said flat bottom wall.

11. The ophthalmic surgical instrument in accordance with claim 1, wherein said enlarged tip further defines a flat top wall opposing said flat bottom wall, wherein a top frustoconical surface connects between said needle shaft portion and said flat top wall, and a bottom frustoconical surface connects between said needle shaft portion and said flat bottom wall.

12. The ophthalmic surgical instrument in accordance with claim 1, wherein said enlarged tip further defines a flat top wall opposing said flat bottom wall, and wherein said flat top wall defines a first thickness and said flat bottom wall defines a second thickness that is greater than said first thickness.

13. The ophthalmic surgical instrument in accordance with claim 1, wherein said enlarged tip further defines a convexly curved top wall opposing said flat bottom wall, and wherein said convexly curved top wall defines a first thickness and said flat bottom wall defines a second thickness that is less than said first thickness.

14. The ophthalmic surgical instrument in accordance with claim 1, further comprising a proximal end of said needle shaft portion removably attached to a vibratory handpiece.

15. An ophthalmic surgical instrument, said instrument comprising:
a hollow needle shaft portion defining a longitudinal shaft axis and having an internal aspiration passage; and
an enlarged tip joined to a distal end of said needle shaft portion,
said enlarged tip defining an open mouth communicating with said aspiration passage, said enlarged tip defining a convexly curved top surface connecting to said needle shaft portion, and said enlarged tip defining an annular flat end face disposed at an acute angle relative to a plane extending normally through said longitudinal shaft axis.

16. The ophthalmic surgical instrument in accordance with claim 15, wherein said acute angle is about 5 degrees.

17. The ophthalmic surgical instrument in accordance with claim 15, said enlarged tip defines a bottom frustoconical surface connecting to said needle shaft portion.

18. The ophthalmic surgical instrument in accordance with claim 17, wherein said bottom frustoconical surface extends distally relative to said convexly curved top surface, along a tip axis, such that said open mouth is disposed at an acute angle relative to a plane extending normally through said tip axis.

19. The ophthalmic surgical instrument in accordance with claim 15, further comprising a proximal end of said needle shaft portion removably attached to a vibratory handpiece.

20. An ophthalmic surgical instrument, said instrument comprising:
a hollow needle shaft portion defining a longitudinal shaft axis and having an internal aspiration passage; and
an enlarged tip joined to a distal end of said needle shaft portion,
said enlarged tip defining an open mouth communicating with said aspiration passage, said enlarged tip defining a convexly curved top wall defining a first radius of curvature, said enlarged tip further defining an opposing flat bottom wall located between two convexly curved side walls defining a second radius of curvature.

21. The ophthalmic surgical instrument in accordance with claim 20, wherein said first radius of curvature is greater than said second radius of curvature.

22. (canceled)

23. (canceled)

24. (canceled)

25. The ophthalmic surgical instrument in accordance with claim 20, wherein said convexly curved top wall extends a greater distance along said longitudinal axis than said flat bottom wall.

26. (canceled)

27. The ophthalmic surgical instrument in accordance with claim 20, wherein said convexly curved top wall defines a first thickness and said flat bottom wall defines a second thickness that is greater than said first thickness.

28.-53. (canceled)
